(19) United States
(12) Patent Application Publication
(10) Pub. No.: US 2020/0237341 A1
(43) Pub. Date: Jul. 30, 2020

(54) Medical Ultrasound system using transducer with circular formation elements placement and built-in MEMS motion sensors

(71) Applicants: Jin HAO, Irvine, CA (US); Zhongyu Yang, San Diego, CA (US)

(72) Inventors: Jin HAO, Irvine, CA (US); Zhongyu Yang, San Diego, CA (US)

(21) Appl. No.: 16/258,628

(22) Filed: Jan. 27, 2019

(51) Int. Cl. A61B 8/00; A61B 8/08; A61B 8/06

(52) U.S. Cl. CPC A61B 8/4444; A61B 8/4483; B81B 2201/02; A61B 8/06; A61B 8/488

(57) ABSTRACT

A Medical Ultrasound System with circular formation elements placement ultrasonic transducer. Each element of transducer generates the ultrasonic signal to human or animal body. Receiving signal from the transducer elements on same circle is directly connected and summed. The summed signal of each element circle is independently delayed by the digital controlled delay and then summed by the adder. The final summed signal after the adder is sent to Post Processing Unit. Post Processing Unit processes the final summed signal for the medical diagnosis, and the beam line is from the center of the circular plane of the transducer elements formation and perpendicular to this plane. The ultrasonic transducer has MEMS based motion sensors: accelerometer-sensors and gyro-sensors built in. The MEMS based motion sensors send the real-time transducer location and angle information to Post Processing Unit. In PW Mode, Post Processing Unit detects and calculates the direction of blood vessel by finding the center of largest distribution area of non-zero magnitude of Doppler shift frequency on the Beam line, the blood vessel direction and angle are known. The physical blood flow speed in the blood vessel is automatically calculated by the measured Doppler shift frequency and the angle between the direction of the blood vessel and the direction of the transducer beam line. In B Mode, Post Processing Unit is splicing the beam lines by the location and angle information from the MEMS sensors during the movement of the transducer.

The present invention relates to the medical ultrasound system, and in particular, to the medical ultrasound system with circular formation elements placement transducer, is to do automatic blood speed diagnosis and B-Mode Image creation by ultrasonic transducer with built-in MEMS motion sensors.

### BACKGROUND OF THE INVENTION

Ultrasonic transducer is widely used for the diagnostic of human and animal. The transducer transmits acoustic waves to target, receives the echo back sound waves. The acquired real-time information of the echo back sound waves is being processed and presented for medical professional for further diagnostic purpose.

However, the current ultrasonic medical system, due to the complexity design of the transducer and the beam-forming technology, they have effects: 1) High cost for the transducer and whole system, 2) The size of the medical system is large, 3) The medical system needs professional (sonographer and doctor) to operate, 4) Diagnostic image and result need professional to interpret.

Because of limited resource of ultrasonic medical system and sonographer, the portable, low complexity, low cost, automatic result interpret ultrasonic medical system with dedicate designed transducer is needed to be used by families, clinics and hospitals.

### SUMMARY OF THE INVENTION

The present invention provides the Medical Ultrasound System using transducer with circular formation elements placement. And also provides the automatic blood flow speed diagnosis and B-Mode Image creation by ultrasonic transducer with built-in MEMS motion sensors.

The new ultrasound elements formation is circular arranged. All elements on the transducer send the ultrasound signal at the same time along the center beam axis. The echo-back ultrasound signals from elements of the same circle are summed directly. Then summed signals from different circles are delayed and summed to the final signal. The final signal is used to calculate the Doppler shift frequency and the heart beats. The circular formation elements placement ultrasonic transducer has one beam line from the center of the circular plane of transducer elements formation and perpendicular to this plane

With the Micro-Electro-Mechanical Systems (MEMS) based motion sensors: gyro-sensors and accelerometer-sensors built in to the transducer, in Pulsed Wave Doppler Mode (PW Mode) the blood flow speed is automatically calculated.

In B-image Mode (B Mode), with the movement of the transducer, the human and animal body two-dimensional scanned image is automatically generated.

### BRIEF DESCRIPTION OF THE DRAWINGS

FIG. 1 shows a perspective view of the preferred ultrasonic transducer circular formation elements placement setup

FIG. 2 shows the perspective view of the beam-forming and the beam line of the present invention

FIG. 3 shows a preferred embodiment of the overview of the present invention

FIG. 4 shows a preferred embodiment of the present invention with MEMS based motion sensors for automatic blood flow speed calculation in PW Mode

FIG. 5a shows a preferred embodiment of the present invention with MEMS based motion sensors for auto tracing two center points within blood vessel.

FIG. 5b shows a preferred embodiment of the present invention with MEMS based motion sensor for automatic blood vessel angle calculation in PW Mode.

FIG. 6 shows a B Mode image generation of a tested prototype

### DETAILED DESCRIPTION OF THE PREFERRED EMBODIMENTS

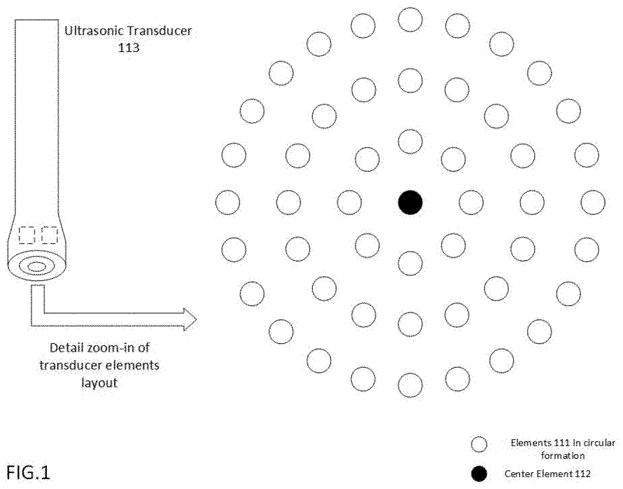

FIG. 1 shows ultrasonic Elements 111 are placed in the circular way on the head of the Ultrasonic Transducer 113. The Center Element 112 is optional. The elements number on each circle can be changed and the number of the circles can be changed as well.

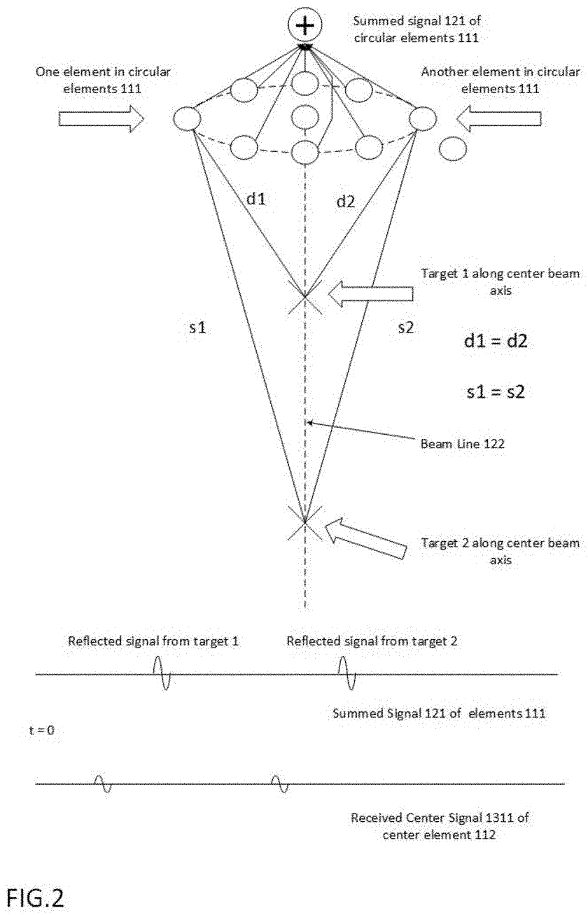

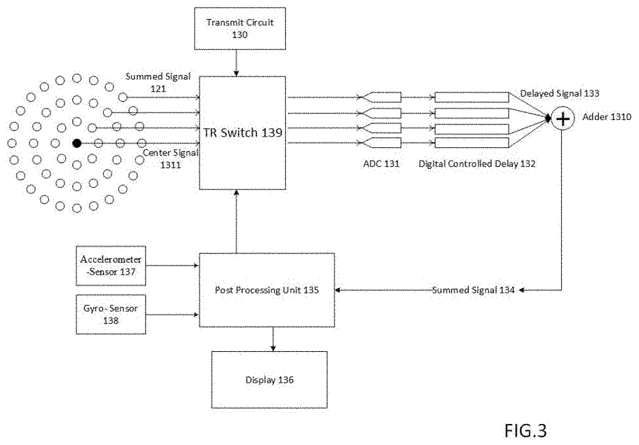

FIG. 2 shows the beam-forming and the beam line of elements 111 in the same circle. The signals from elements 111 in the same circle are connected directly to one summed signal 121 without delay. And the beam line 122 is in the center of the elements formation. Because elements 111 in the same circle can be grouped and summed without delay, compared to the conventional transducer each element needs to be independently delayed and summed, the circular formation elements placement saves the cost and reduces the complexity of the ultrasonic system which is shown in FIG. 3.

FIG. 3 shows the summed signals 121 from different elements circles and the center signal 1311 from center element 112 which send the ultrasonic signals generated by the Transmit Circuit 130 are received to the dedicate Analog to Digital Converter (ADC 131). And after the digital controlled delay 132, the delayed signals 133 from each elements circle and center element are summed by adder 1310. Then the summed signal 134 will go to Post Processing Unit 135 to the Display 136 to show the results. The MEMS based motion sensor accelerometer-sensor 137 and MEMS based motion sensor Gyro-sensor 138 send the real-time motion information of the ultrasonic transducer 113 to Post Processing Unit 135 for physical blood flow speed monitor or B Mode image creation 161.

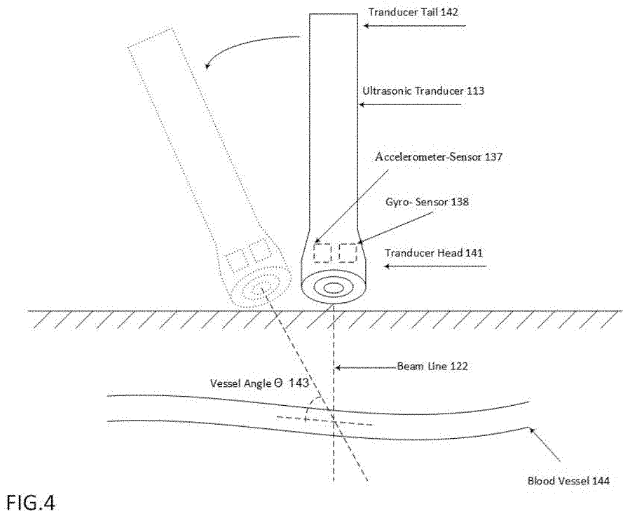

Physical blood flow speed monitor FIG. 4

The physical blood flow speed is calculated in the PW mode.

The physical blood flow speed is the equation below:

$$v = \frac{f * c}{2 * f 0 * cos ( θ )} . \qquad ( 1 )$$

Here f0 is the frequency of the transmit sound wave, c is the sound speed in tissue, Vessel Angle θ 143 is the angle between the direction of beam line 122 and direction of the blood vessel 144, f is the Doppler shift frequency.

The frequency of the transmit wave f0 is known and controlled. The Doppler shift frequency f is known from the receiving signal. To acquire the blood vessel angle θ 143, one accelerometer-sensor 137 and one Gyro-sensor 138 are built in to the transducer head 141 as shown in FIG. 4.

The vessel angle θ 143 is automatically calculated in Post Processing Unit 135 according to the motion information from the Accelerometer-Sensor 137, Gyro-sensor 138 and the receiving ultrasonic summed signal 134.

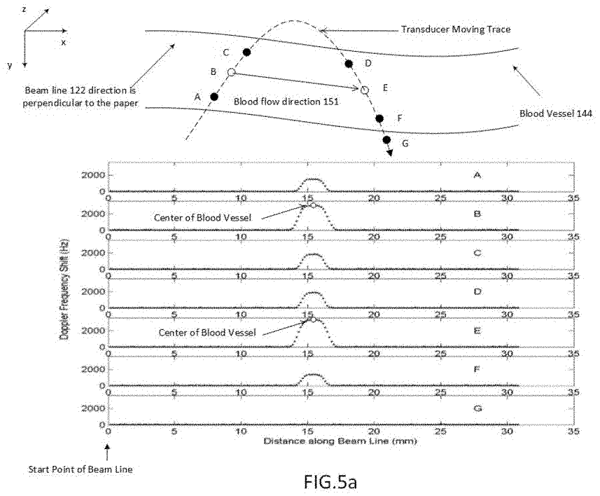

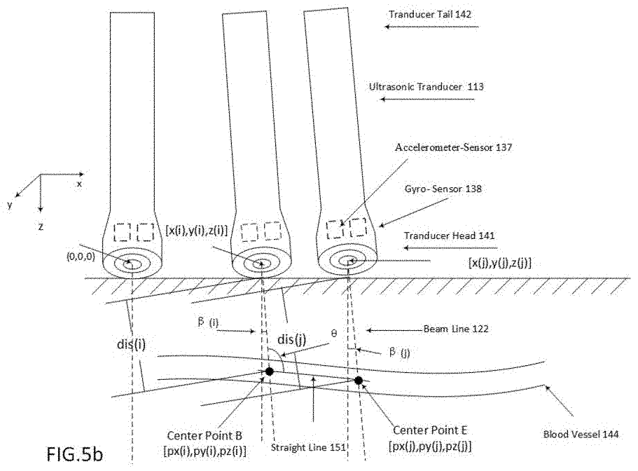

FIG. 5a and FIG. 5b shows the detail measurement and calculation of the Vessel angle θ 143 between the direction of the Beam line 122 and direction of the Blood Vessel 144. FIG. 5a is the top direction view of the FIG. 5b

The overview of the blood Vessel angle θ 143 calculation:

To acquire the blood Vessel angle θ 143, at first the transducer is moved on the x-y surface as shown in FIG. 5a to find the center point locations in the blood vessel, the beam line direction is along z axis. By moving the transducer around, the Magnitude of Doppler shift frequency varies depending on whether the beam intersects with blood flow or not. In PW Mode, the distribution of the Magnitude of Doppler shift frequency on Beam line 122 is automatically calculated and monitored by the Post Processing Unit 135.

Only the echo signal from blood in the Blood Vessel 144 has the non-zero Magnitude of Doppler shift frequency on the Beam Line 122. By moving the Ultrasonic Transducer 113 from location A to location C, the non-zero Magnitude of Doppler shift frequency has the largest distribution area when the Beam line 122 is passing through the center of the blood vessel at Location B as shown in FIG. 5a. Post Processing Unit 135 recognizes the center location of the blood vessel 144 which is the same location in the center of the largest distribution of the non-zero Magnitude of Doppler shift frequency on Beam line 122. The same method applies to the Beam Line 122 moving from Location D to Location F. Then Post Processing Unit recognizes two center locations of the blood vessel 144. By connecting the Location B and Location E to the Straight line 151, the direction of the blood vessel 144 is found.

The coordinates of points B and E are acquired and derived from the MEMS based motion sensors, like accelerometer-sensors 137 and Gyro-sensors 138 as shown in FIG. 5b. The location and direction of the beam line 122 is also known from the MEMS based motion sensors. Then the blood vessel angle θ 143 between the direction of the Straight line 151 and the direction of the Beam line 122 is calculated by the Post Processing Unit 135.

The details of the blood Vessel angle θ 143 calculation:

At beginning, the coordinates of the start point of Beam Line 122 of Ultrasonic Transducer 113 which is in the center of the circular formation of elements 111 is set to (0,0,0) in Post Processing Unit 135. When the transducer is moved along the human body, the acceleration speed aₓ, a_{y} and a_{z} along axis x,y,z and rotating angle speed ωₓ, ω_{y}, ω_{z} are collected from the accelerometer-sensor 137 motion sensor and Gyro-sensor 138 in real-time. The new center positions x_{acc}(i+1) on the x axis of the transdcuer surface are calculated using the parameters recorded by accelerometer-sensor 137. The equations are shown below:

velₓ(i+1)=velₓ(i)+aₓ(i+1)* dt

x_{acc}(i+1)=x_{acc}(i)+velₓ(i+1)* dt+0.5* aₓ(i+1)* dt²

The initial condition velₓ(0)=0 and aₓ(0)=0. And i represents the time interval index after resetting the origin; dt is the time interval acquiring the acceleration parameters. Same rules apply to y and z axis.

The rotation angles along x, y and z axis are defined as α, β, γ. The rotation angle α(i) is calculated using the parameters recorded by Gyro-sensor 138. The equations are shown below:

α(i)=ωₓ(i)* dt

β(i)=ω_{y}(i)*dt

β(i)=ω_{z}(i)*dt

Where i represents the time interval index after resetting the origin, dt is the time interval acquiring the rotation parameters. Then the rotation matrix along x, y and z axis is calculated using

$${R}_{x} ( α ) = [ \begin{matrix}1 & 0 & 0 \\ 0 & cos ( α ) & - sin ( α ) \\ 0 & sin ( α ) & cos ( α )\end{matrix} ]$$

$${R}_{y} ( β ) = [ \begin{matrix}cos ( β ) & 0 & sin ( β ) \\ 0 & 1 & 0 \\ - sin ( β ) & 0 & cos ( β )\end{matrix} ]$$

$${R}_{z} ( γ ) = [ \begin{matrix}cos ( γ ) & - sin ( γ ) & 0 \\ sin ( γ ) & cos ( γ ) & 0 \\ 0 & 0 & 1\end{matrix} ]$$

Then the final coordinates of center of the elements formation which is on the transducer surface are calculated using

$$[ \begin{matrix}x ( i ) \\ y ( i ) \\ z ( i )\end{matrix} ] = {R}_{x} ( α ) {R}_{y} ( β ) {R}_{z} ( γ ) [ \begin{matrix}{x}_{acc} ( i ) \\ {y}_{acc} ( i ) \\ {z}_{acc} ( i )\end{matrix} ]$$

At each new position [x(i),y(i),z(i)], beam line 122 is acquired repeated at the preset frequency which is controlled by Post Processing Unit 135. The sample location (dis(i)) of the center point B in the blood vessel is extracted to calculate the blood flow center point coordinates [px(i), py(i), pz(i)] using the following equation.

$$[ \begin{matrix}px ( i ) \\ py ( i ) \\ pz ( i )\end{matrix} ] = {R}_{x} ( α ) {R}_{y} ( β ) {R}_{z} ( γ ) [ \begin{matrix}{x}_{acc} ( i ) \\ {y}_{acc} ( i ) \\ {z}_{a c c} ( i ) + dis ( i )\end{matrix} ]$$

When the transducer is moved to the center position E, applying the same rule, another blood flow center point coordinates [px(j), py(j), pz(j)] are acquired. Then the direction vector of the blood flow [symbol] is calculated as

[symbol]

=[px(j),py(j),pz(j)]−[px(i),py(i),pz(i)]

And the direction vector of the beam line z,28 is calculated as

[symbol]

=[x(i),y(i),z(i)]−[px(i),py(i),pz(i)]

Finally the blood vessel angle between the direction of the beam line and the direction of the blood flow is:

$$cos ( θ ) = \frac{\vec{u} \cdot \vec{v}}{\vert \vec{u} \vert \vert \vec{v} \vert}$$

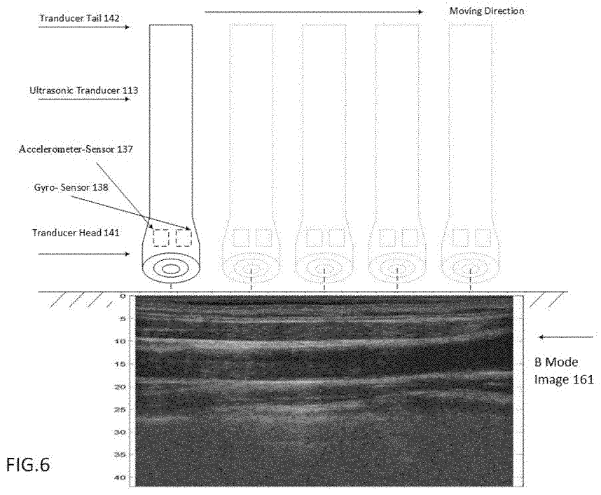

The B mode image is generated according to the motion information from the MEMS motion sensor accelerometer-sensor 137, Gyro-sensor 138 as FIG. 6. By moving of the ultrasonic transducer 113, the location and angle information of the motion sensors are used by Post Processing Unit 135. The beam line 122 information is splicing together to form a B-Mode Image 161.

Although the above-preferred embodiments have been described with specificity, persons skilled in this art will recognize that many changes to the specific embodiments disclosed above could be made without departing from the spirit of the invention. For example, although FIG. 1 has the center element 112, this center element can also be removed. FIG. 2 has three circles of the elements; the circle number could also be two, four, or any other numbers except zero. FIG. 3 has one piece of Gyro-sensor and one piece of accelerometer-sensor; the number of sensors could also be two, three or more; the type of sensor can also be other types as far as it can send the location and angle related signal to the Post Processing Unit 135. And the method of automatic blood flow speed calculation can not only be implemented by the circular formation elements formation based transducer, it can also be implemented by any other type of ultrasonic transducer with built-in MEMS motion sensors.

Therefore, the attached claims and their legal equivalents should determine the scope of the invention

What is claimed is:

1) The medical ultrasound system, comprising
A) Circular formation elements placement ultrasonic transducer for ultrasonic signal transmitting and receiving
B) The transducer built-in MEMS motion sensors, accelerometer-sensors and gyro-sensors for transducer location and angle information.
C) Transmitting and Receiving Circuit, Analog to digital converters, Digital controlled delay, Adder, Post Processing Unit for automatic blood flow speed calculation, heart beat monitoring, and B-Mode image creation.

2) The medical ultrasound system as in claim 1, wherein said the circular formation elements placement ultrasonic transducer is comprising:
A) Circular formation elements for ultrasonic signal transmitting and receiving
B) Center element for ultrasonic signal transmitting and receiving

3) The medical ultrasound system as in claim 1, wherein said ultrasonic signals which are received from ultrasonic transducer elements on the same circle are summed together for beam-forming the beam line along the axis which is passing through the center of the circular plane of transducer elements formation and perpendicular to this plane.

4) The medical ultrasound system as in claim 3, wherein said each summed elements circle signal from transducer is independently delayed by Digital controlled delay.

5) The medical ultrasound system as in claim 4, wherein said each delayed element circle signal from the digital controlled delay is summed by Adder for beam-forming the beam line.

6) The medical ultrasound system as in claim 5, wherein said the summed signal after the Adder is processed by the Post Processing Unit to calculate and monitor the distribution of magnitude of Doppler shift frequency on the Beam line in PW mode.

7) The medical ultrasound system as in claim 6, wherein said only the blood in the blood vessel has non-zero magnitude of Doppler Shift frequency

8) The medical ultrasound system as in claim 7, wherein said the MEMS based motion sensors, accelerometer-sensors and gyro-sensors provide the direction and location of the beam line to Post Processing Unit.

9) The medical ultrasound system as in claim 8, wherein said the Post Processing Unit finds the locations of the center of the blood vessel by finding the center of largest distribution area of non-zero magnitude of Doppler shift frequency on the Beam line.

10) The medical ultrasound system as in claim 9, wherein said the two locations of the center of the blood vessel are connected by straight line to form the direction of the blood vessel

11) The medical ultrasound system as in claim 10, wherein said the blood vessel angle is determined by the direction of blood vessel and the present direction of the beam line.

12) The medical ultrasound system as in claim 11, wherein said Post Processing Unit automatically calculates the physical blood flow speed determined by the blood vessel angle and the currently measured magnitude of Doppler shift frequency.

13) The medical ultrasound system as in claim 1, wherein said the location information of beam line is provided by the MEMS based accelerometer-sensors and gyro-sensors which are built in the transducer

14) The medical ultrasound system as in claim 13, wherein said Post Processing Unit creates B-Mode image by splicing the different locations of the beam line of the moving transducer based on the location information.
